(19) United States
(12) Patent Application Publication
(10) Pub. No.: US 2023/0034120 A1
(43) Pub. Date: Feb. 2, 2023

(54) ENDOSCOPE CAP

(71) Applicant: Jihwan KO, Busan (KR)

(72) Inventor: Jihwan KO, Busan (KR)

(21) Appl. No.: 17/459,532

(22) Filed: Aug. 27, 2021

(30) Foreign Application Priority Data
Jul. 28, 2021 (KR) 10-2021-0099400

(51) Int. Cl. A61B 1/00

(52) U.S. Cl. CPC A61B 1/00137; A61B 1/00094

(57) ABSTRACT

Disclosed is an endoscope cap includes a cap body coupled to a tip end of an endoscope introduced into an interior of an organism, an expansion/contraction part provided in the cap body, and expanded or contracted through injection of air, and an air supply part that injects or suctions the air into or from the expansion/contraction part, and the air supply part expands the expansion/contraction part by injecting the air into the expansion/contraction part and the expanded expansion/contraction part bends back a target tissue in the interior of the organism when the cap body reaches the target tissue.

### CROSS-REFERENCE TO RELATED APPLICATIONS

A claim for priority under 35 U.S.C. § 119 is made to Korean Pat. Application No. 10-2021-0099400 filed on Jul. 28, 2021 in the Korean Intellectual Property Office, the entire contents of which are hereby incorporated by reference.

### BACKGROUND

Embodiments of the inventive concept described herein relate to an endoscope cap.

In general, an endoscope is a medical instrument that is inserted into an interior of an organism to observe a state of the interior of the organism, and has contributed to medical development until now. A polyp in the interior of the organism may be discovered by using the endoscope.

However, the interior of the organism has winkle sections and superimposition sections, and when a polyp is hidden in a deep part of a wrinkle section or a superimposition section, it is difficult to find out the polyp by using an endoscope.

### Prior Technical Documents

### Pat. Documents

(Pat. Document 1) Korean Pat. Application Publication No. 10-1651147 (Aug. 19, 2016)

### SUMMARY

Embodiments of the inventive concept provide an endoscope cap that allows a diseased part hidden in a target tissue in an interior of an organism to be easily revealed by bending back the target tissue in the interior of the organism during an endoscope inspection.

The technical objects of the inventive concept are not limited to the above-mentioned ones, and the other unmentioned technical objects will become apparent to those skilled in the art from the following description.

According to an embodiment, an endoscope cap includes a cap body coupled to a tip end of an endoscope introduced into an interior of an organism, an expansion/contraction part provided in the cap body, and expanded or contracted through injection of air, and an air supply part that injects or suctions the air into or from the expansion/contraction part, and the air supply part expands the expansion/contraction part by injecting the air into the expansion/contraction part and the expanded expansion/contraction part wets a target tissue in the interior of the organism when the cap body reaches the target tissue.

Furthermore, the endoscope cap may include a manifold connected to the air supply part and provided in the cap body, a plurality of expansion/contraction parts may be provided, and the plurality of expansion/contraction parts may be connected to the manifold, and the plurality of expansion/contraction part may be simultaneously expanded when the air is injected into the manifold by the air supply part.

Furthermore, the plurality of expansion/contraction parts may be disposed radially along a circumference of the manifold.

Furthermore, ends of the expansion/contraction parts may be connected to a tip end of the manifold, the expansion/contraction parts may be folded in a direction, in which the endoscope is discharged from the interior of the organism when the expansion/contraction parts are contracted, and the expansion/contraction parts may be folded in a direction, in which the endoscope is introduced into the interior of the organism when the expansion/contraction parts are expanded.

Furthermore, the cap body may be screw-coupled to the tip end of the endoscope.

Furthermore, a tip end of the cap body may be opened.

Detailed items of the other embodiments are included in the detailed description and the accompanying drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating an endoscope cap according to an embodiment of the inventive concept;

FIG. 2 is an exploded perspective view illustrating an endoscope cap according to an embodiment of the inventive concept;

FIG. 3 is a perspective view illustrating an operation process of an endoscope cap according to an embodiment of the inventive concept;

FIG. 4 is a schematic view illustrating a process of bending back a target tissue in an interior of an organism by an endoscope cap according to an embodiment of the inventive concept; and

FIG. 5 is an exploded perspective view illustrating an endoscope cap according to another embodiment of the inventive concept.

### DETAILED DESCRIPTION

The above and other aspects, features, and advantages of the inventive concept will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains, and the inventive concept will be defined by the scope of the claims.

The terms used herein are provided to describe the embodiments but not to limit the inventive concept. In the specification, the singular forms include plural forms unless particularly mentioned. The terms "comprises" and/or "comprising" used herein does not exclude presence or addition of one or more other elements, in addition to the aforementioned elements. Throughout the specification, the same reference numerals denote the same elements, and "and/or" includes the respective elements and all combinations of the elements. Although "first", "second" and the like are used to describe various elements, the elements are not limited by the terms. The terms are used simply to distinguish one element from other elements. Accordingly, it is apparent that a first element mentioned in the following may be a second element without departing from the spirit of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, exemplary embodiments of the inventive concept will be described in detail with reference to the accompanying drawings.

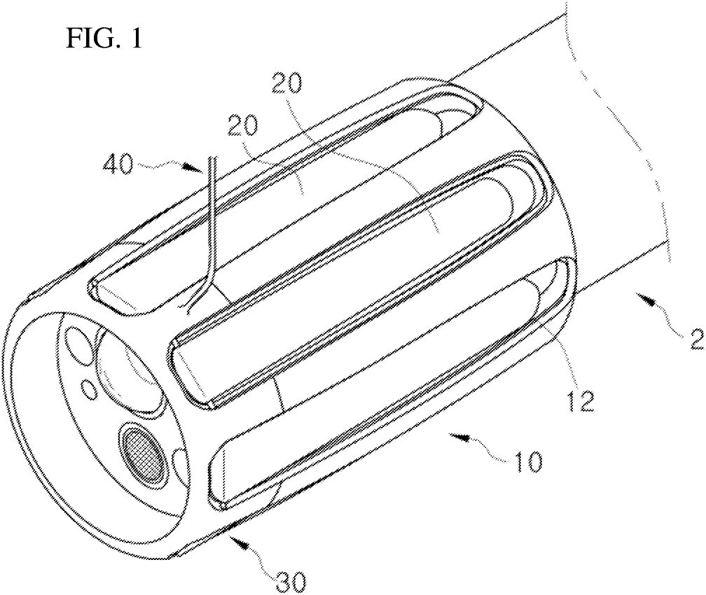

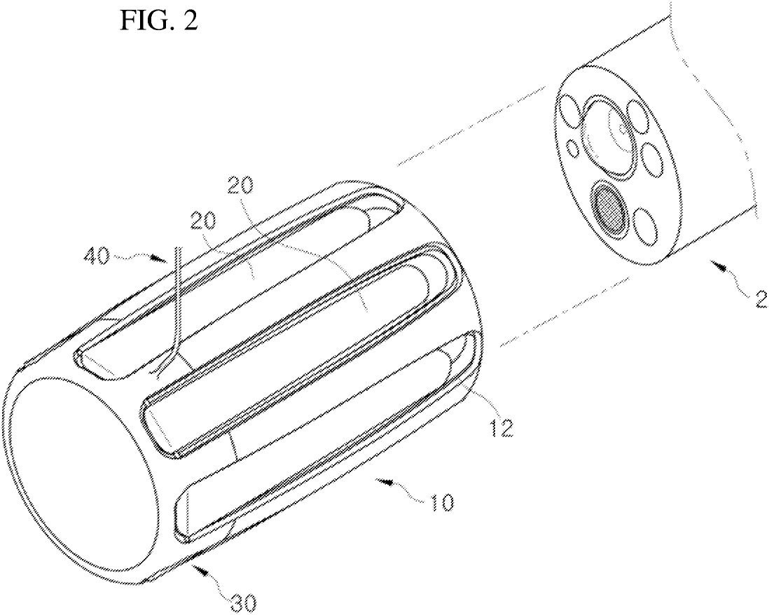

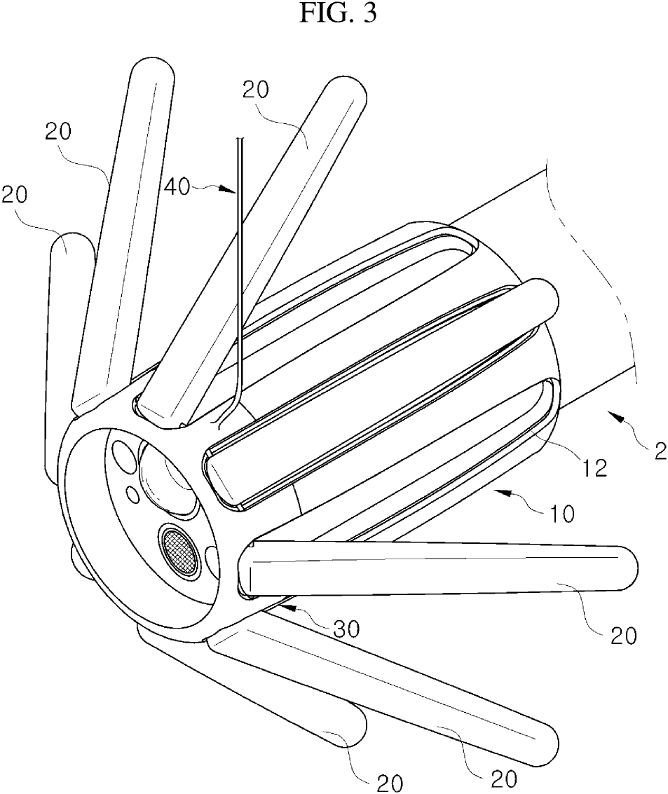

FIG. 1 is a perspective view illustrating an endoscope cap according to an embodiment of the inventive concept. FIG. 2 is an exploded perspective view illustrating an endoscope cap according to an embodiment of the inventive concept. FIG. 3 is a perspective view illustrating an operation process of an endoscope cap according to an embodiment of the inventive concept.

As illustrated in FIGS. 1 and 2, an endoscope cap according to an embodiment of the inventive concept includes a cap body 10, an expansion/contraction part 20, and an air supply part 30.

The cap body 10 is a basic body of the inventive concept, and functions to cover a tip end of an endoscope 2. The cap body 10 may be coupled to the tip end of the endoscope 2 introduced into an interior of an organism. Here, the cap body 10 may be coupled to the tip end of the endoscope to protrude from the tip end of the endoscope 2 in a direction, in which the endoscope 2 is introduced into the interior of the organism. That is, the cap body 10 may be coupled to protrude from the tip end of the endoscope 2.

The cap body 10 may have a cylindrical shape. A hollow portion may be formed in an interior of the cap body 10 along a lengthwise direction thereof, and the tip end of the cap body 10 may be opened. Accordingly, the endoscope 2 may photograph the interior of the organism through the opening of the cap body.

The cap body 10 may have a diameter that is larger than the tip end of the endoscope 2. The cap body 10 may be coupled to the tip end of the endoscope 2 to be stepped. Accordingly, when the cap body 10 reaches a target tissue 1 in the interior of the organism, the step of the cap body 10 may bend back the target tissue 1 in the interior of the organism, but in the inventive concept, the expansion/contraction part 20, which will be described below, mainly functions to bend k the target tissue 1 in the interior of the organism.

The expansion/contraction part 20 may be provided in the cap body 10 to be expanded and contracted through injection of air, and the injection of the air may be performed by the air supply part 30, which will be described below. Here, the expansion/contraction part 20 may be seated on a recessed part 12 that is recessed on an outer surface of the cap body 10. Accordingly, when the cap body 10 is introduced into the interior of the organism, the expansion/contraction part 20 may be seated in the recessed part 12 of the cap body 10 whereby an inner wall of the organism may be prevented from being stimulated.

The expansion/contraction part 20 functions to bend back the target tissue 1 in the interior of the organism. For example, when the expansion/contraction part 20 reaches the target tissue 1 in the interior of the organism, the air supply part 30 expands the expansion/contraction part 20 by injecting the air into the expansion/contraction part 20, and the expanded expansion/contraction part 20 may bend back the target tissue in the interior of the organism.

Meanwhile, selectively, the expanded expansion/contraction part 20 may bend back the target tissue 1 in the interior of the organism while moving in a direction in which it is introduced into the organism or in a direction in which it is discharged from the interior of the organism. Then, as the tip end of the endoscope 2 moves, the expanded expansion/contraction part 20 may move.

Meanwhile, the expansion/contraction part 20 may be a balloon, a tube, or the like.

The air supply part 30 functions to inject or suction the air into or from the expansion/contraction part 20. The air supply part 30 may be a pump that injects or suction the air. The air supply part 30 may contract the expansion/contraction part 20 by suctioning the air in the expansion/contraction part 20 before the expansion/contraction part 20 reaches the target tissue 1 in the interior of the organism, and may expand the expansion/contraction part 20 by injecting the air into the expansion/contraction part 20 after the expansion/contraction part 20 reaches the target tissue 1 in the interior of the organism.

The endoscope cap according to the embodiment of the inventive concept may further include a manifold 40.

The manifold 40 functions to deliver the air supplied from the air supply part 30 to the expansion/contraction part 20. The manifold 40 may be connected to the air supply part 30 and may be provided at the tip end of the cap body 10.

The manifold 40 may have an annular shape. A stay space, in which the air stays, may be provided in the interior of the manifold 40, and a connection shaft, to which the expansion/contraction part 20 is connected, may be formed at the tip end of the manifold 40. Then, a plurality of connection shafts may be provided, and the plurality of connection shafts may be disposed radially at the tip end of the manifold 40.

Here, a plurality of expansion/contraction parts 20 may be provided, and the plurality of expansion/contraction parts 20 may be connected to a plurality of communication holes of the manifold 40, respectively. Accordingly, when the air is injected from the air supply part 30 into the manifold 40, the plurality of expansion/contraction parts 20 may be simultaneously expanded.

The plurality of expansion/contraction parts 20 may be disposed radially along a circumference of the manifold 40. Accordingly, when the air is injected from the air supply part 30 into the manifold 40, the plurality of expansion/contraction parts 20 may be simultaneously expanded radially.

Ends of the expansion/contraction parts 20 may be connected to the tip end of the manifold 40, and opposite ends of the expansion/contraction parts 20 may be folded to a rear end of the manifold 40. Accordingly, when the expansion/contraction parts 20 are contracted, the expansion/contraction parts 20 may be folded in a direction, in which the endoscope 2 is discharged from the interior of the organism. When the contracted expansion/contraction parts 20 are introduced into the interior of the organism, they move along the inner wall of the organism, and thus the inner wall may be prevented from being stimulated.

Accordingly, when the expansion/contraction parts 20 are expanded, the expansion/contraction parts 20 may be unfolded in a direction, in which the endoscope 2 is introduced into the interior of the organism. The expanded expansion/contraction parts 20 may wet the target tissue 1 in the interior of the organism.

Meanwhile, depending on an amount or a pressure of the air injected into the expansion/contraction parts 20, an unfolding angle, by which the expansion/contraction parts 20 are unfolded from the endoscope cap 2, may be adjusted. Accordingly, the endoscope 2 may be used while the expansion/contraction parts 20 are deformed into various shapes according to a shape of the interior of the organism.

Hereinafter, an operation of the endoscope cap according to the embodiment of the inventive concept will be described.

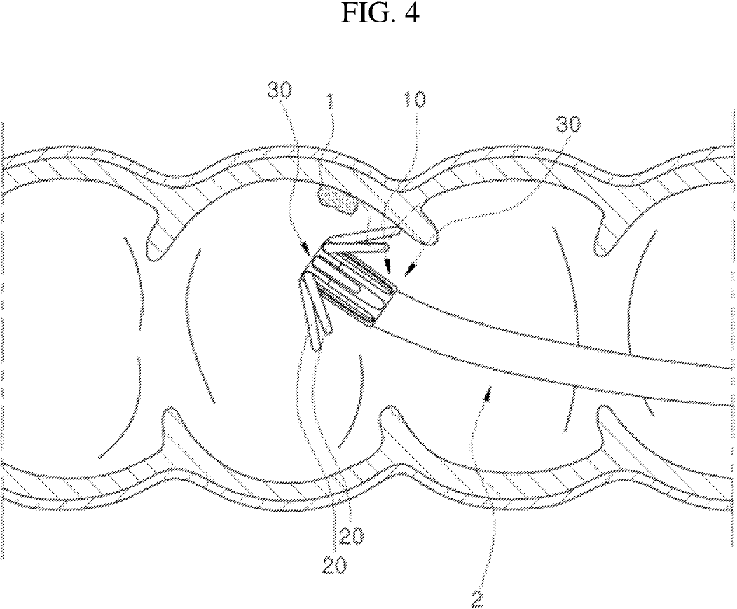

FIG. 4 is a schematic view illustrating a process of bending back a target tissue 1 in an interior of an organism by an endoscope cap according to an embodiment of the inventive concept.

As illustrated in FIG. 4, first, the cap body 10 is introduced into the interior of the organism. Then, the air supply part 30 suctions the air in the expansion/contraction part 20, and the contracted expansion/contraction part 20 is introduced along the inner wall of the organism.

Next, when the cap body 10 reaches the target tissue 1 in the interior of the organism, the air supply part 30 expands the expansion/contraction part 20 by injecting the air into the expansion/contraction part 20. As a result, because the expanded expansion/contraction part 20 wets the target tissue 1 in the interior of the organism, a diseased part hidden in the target tissue 1 in the interior of the organism may be revealed to the endoscope 2.

Meanwhile, selectively, the expanded expansion/contraction part 20 may wet the target tissue 1 in the interior of the organism while moving in a direction in which it is introduced into the organism or in a direction in which it is discharged from the interior of the organism.

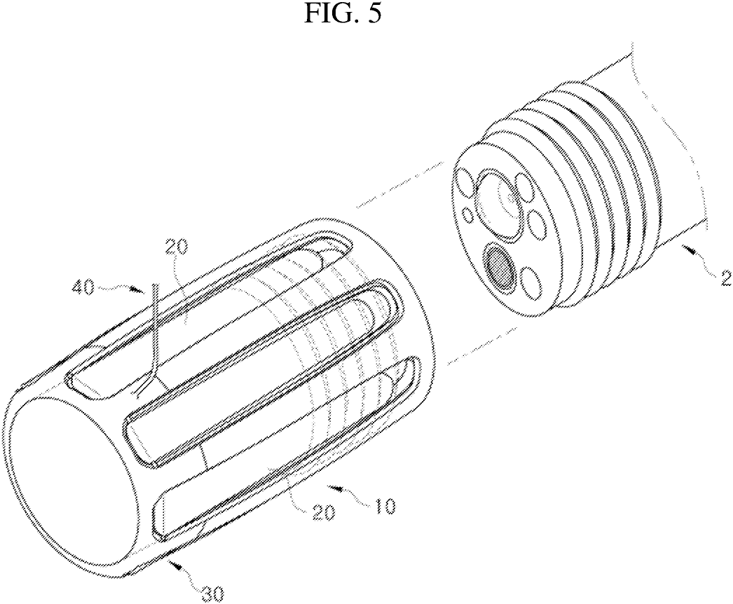

FIG. 5 is an exploded perspective view illustrating an endoscope cap according to another embodiment of the inventive concept.

As illustrated in FIG. 5, the cap body 10 may be screw-coupled to the tip end of the endoscope 2. Here, the cap body 10 may be rotated by a driving device, and the cap body 10 may move along the tip end of the endoscope 2 when the cap body 10 rotates forwards.

Accordingly, even when the cap body 10 is coupled to the tip end of the endoscope 2, the cap body 10 may move along the tip end of the endoscope 2, and thus the expansion/contraction part 20 coupled to the cap body 10 may wet the target tissue 1 in the interior of the organism while moving together with the cap body 10.

According to the inventive concept, the endoscope cap according to the embodiment of the inventive concept allows the diseased part hidden in the target tissue 1 in the interior of the organism to be easily revealed to the endoscope 2 by bending back the target tissue 1 in the interior of the organism during an inspection using the endoscope 2.

The endoscope cap according to the embodiment of the inventive concept allows a diseased part hidden in a target tissue in an interior of an organism to be easily revealed by bending back the target tissue in the interior of the organism during an endoscope inspection.

The effects of the inventive concept are not limited thereto, and other unmentioned effects of the inventive concept may be clearly appreciated by those skilled in the art from the following descriptions.

Although the exemplary embodiments of the inventive concept have been described with reference to the accompanying drawings, it will be understood by those skilled in the art to which the inventive concept pertains that the inventive concept can be carried out in other detailed forms without changing the technical spirits and essential features thereof. Therefore, the above-described embodiments are exemplary in all aspects, and should be construed not to be restrictive.

What is claimed is:

1. An endoscope cap comprising:
a cap body coupled to a tip end of an endoscope introduced into an interior of an organism;
an expansion/contraction part provided in the cap body, and expanded or contracted through injection of air; and
an air supply part configured to inject or suction the air into or from the expansion/contraction part,
wherein the air supply part expands the expansion/contraction part by injecting the air into the expansion/contraction part and the expanded expansion/contraction part bends back a target tissue in the interior of the organism when the cap body reaches the target tissue.

2. The endoscope cap of claim 1, further comprising:
a manifold connected to the air supply part and provided in the cap body,
wherein a plurality of expansion/contraction parts are provided, and the plurality of expansion/contraction parts are connected to the manifold, and
wherein the plurality of expansion/contraction part are simultaneously expanded when the air is injected into the manifold by the air supply part.

3. The endoscope cap of claim 2, wherein the plurality of expansion/contraction parts are disposed radially along a circumference of the manifold.

4. The endoscope cap of claim 2, wherein ends of the expansion/contraction parts are connected to a tip end of the manifold,
wherein the expansion/contraction parts are folded in a direction, in which the endoscope is discharged from the interior of the organism when the expansion/contraction parts are contracted, and
wherein the expansion/contraction parts are folded in a direction, in which the endoscope is introduced into the interior of the organism when the expansion/contraction parts are expanded.

5. The endoscope cap of claim 1, wherein the cap body is screw-coupled to the tip end of the endoscope.

6. The endoscope cap of claim 1, wherein a tip end of the cap body is opened.
